(19) United States
(12) Patent Application Publication
(10) Pub. No.: US 2026/0263046 A1
(43) Pub. Date: Sep. 10, 2026

(54) Ultrasound-Based Elastography Through Shear-Wave Measurement

(71) Applicant: FUJIFILM SonoSite, Inc., Bothell, WA (US)

(72) Inventors: John Ross Williams, Toronto (CA); Alex Forbrich, Toronto (CA); Cassidy Rose, Toronto (CA); Chenxi Yin, Richmond Hill (CA); Lauren Wirtzfeld, Thornhill (CA); William Andrew Needles, Toronto (CA)

(73) Assignee: FUJIFILM SonoSite, Inc., Bothell, WA (US)

(21) Appl. No.: 19/074,391

(22) Filed: Mar. 9, 2025

Related U.S. Application Data
(60) Provisional application No. 63/563,823, filed on Mar. 11, 2024.

(51) Int. Cl. A61B 8/00; G16H 40/63

(52) U.S. Cl. CPC A61B 8/485; A61B 8/461; G16H 40/63

(57) ABSTRACT

Systems, devices, and methods for ultrasound-based elastography through shear-wave measurement are described. In some implementations, an ultrasound system generates ultrasound data from an ultrasound scan of an anatomy, such as a bodily structure of an organism. The system generates one or more push pulses to induce one or more corresponding shear waves in the anatomy being scanned. The one or more shear waves are scanned by the system, producing shear wave ultrasound data, which is used by the system to determine one or more features of the anatomy being scanned (speed-of-sound, elasticity of the anatomy, etc.). According to some embodiments, the one or more features of the anatomy are further based on the ultrasound data.

### CROSS-REFERENCE TO RELATED APPLICATION(S)

This application claims the benefit of U.S. Provisional Patent Application Ser. No. 63/563,823, filed on Mar. 11, 2024, the disclosure of which is incorporated by reference herein in its entirety.

### BACKGROUND

Tissue elasticity can be indirectly determined based on shear-wave speed measurements. Shear waves can be created in an anatomy by inducing the shear wave via ultrasound push pulses, such as push pulses incident in a tissue of a patient, which exploit acoustic radiation force. The shear wave can be a displacement of matter, where the shear wave causes particle motion in a direction perpendicular to the direction of motion of an energy wave propagating in the matter. The shear wave can, in aspects, be tracked via ultrasound to determine a shear-wave velocity, and tissue elasticity can, in aspects, be determined from the shear-wave velocity, such as via Young's modulus. However, when the shear wave is tracked using ultrafast plane wave imaging, the signal-to-noise ratio (SNR) can be low at greater depths, causing poor sensitivity and corrupting the determined elasticity values.

Further, respiratory and cardiac pulsations can also induce corruption of the measurement due to out-of-plane motion and tissue compression/expansion. The tissue compression/expansion can be difficult to control for, such as in small animals due to their increased rates in relation to acquisition durations, when imaging in real-time.

### SUMMARY

Systems, devices, and methods for ultrasound-based elastography through shear-wave measurement are described. In some implementations, an ultrasound system generates ultrasound data from an ultrasound scan of an anatomy, such as a bodily structure of an organism. The system generates one or more push pulses to induce one or more corresponding shear waves in the anatomy being scanned. The one or more shear waves are scanned by the system, producing ultrasound data to track shear wave propagation, which is used by the system to determine one or more features of the anatomy being scanned (shear wave speed, elasticity of the anatomy, etc.). According to some embodiments, the one or more features of the anatomy are further based on the ultrasound data. The ultrasound-based elastography through shear-wave measurement system can increase the reliability of elasticity measurements in comparison to measurements by conventional ultrasound systems.

In some aspects, an ultrasound system for ultrasound-based elastography through shear-wave measurement is disclosed. The ultrasound system includes an ultrasound scanner, one or more processors in communication with the ultrasound scanner, and one or more computer-readable storage media. The ultrasound scanner is configured to generate ultrasound data based on reflections of ultrasound signals transmitted by the ultrasound scanner at an anatomy. The ultrasound scanner is further configured to generate a first push pulse at a first point within a region of interest (ROI), where the ROI is a region of the anatomy and the first push pulse is configured to induce a first shear wave within the anatomy. The one or more computer-readable storage media have instructions stored thereon that, responsive to execution by the one or more processors, cause the one or more processors to determine, based on the first push pulse, a first velocity of the first shear wave, and determine, based on the first velocity, a first elasticity of the anatomy at the first point.

In some aspects, an ultrasound device for ultrasound-based elastography through shear-wave measurement is disclosed. The ultrasound device includes a housing, an ultrasound scanner coupled to the housing, one or more processors coupled to the housing, the one or more processors in communication with the ultrasound scanner, and one or more computer-readable storage media coupled to the housing. The ultrasound scanner is configured to generate ultrasound data based on reflections of ultrasound signals transmitted by the ultrasound scanner at an anatomy and generate a first push pulse at a first point within an ROI. The one or more computer-readable storage media have instructions stored thereon that, responsive to execution by the one or more processors, cause the one or more processors to determine, based on the first push pulse, a first velocity of the first shear wave; and determine, based on the first velocity, a first elasticity of the anatomy at the first point.

In some aspects, a method for ultrasound-based elastography through shear-wave measurement is disclosed. The method includes receiving first ultrasound data based on reflections of ultrasound signals transmitted by an ultrasound scanner at an anatomy. The first ultrasound data is based on a first push pulse transmitted by the ultrasound scanner at a first point within an ROI. The method further includes determining, by one or more processors and based on the first push pulse, a first velocity of the first shear wave. The method also includes determining, by the one or more processors and based on the first velocity, a first elasticity of the anatomy at the first point.

Other systems, machines, and methods to provide ultrasound-based elastography through shear-wave measurement are also described.

### BRIEF DESCRIPTION OF THE DRAWINGS

The appended drawings illustrate examples and are, therefore, exemplary embodiments and not considered to be limiting in scope.

FIG. 1 illustrates an example environment for an ultrasound-based elastography through shear-wave measurement system in accordance with one or more implementations.

FIG. 2 illustrates an example implementation of the ultrasound machine from FIG. 1.

FIG. 3 illustrates a plot of a shear-wave measurement made by an ultrasound system or device for ultrasound-based elastography through shear-wave measurement.

FIG. 4 illustrates a plot showing a measurement of the shear wave of FIG. 3 after a greater time has elapsed.

FIG. 5 illustrates an example shear-wave velocity map with a region of interest.

FIG. 6 depicts a workflow for determining example maps for overlay and user presentation.

FIG. 7 illustrates an example controller to adjust one or more properties of the push pulses.

FIG. 8 represents an example machine-learning architecture used to train a machine-learned model for ultrasound-based elastography through shear-wave measurement.

FIG. 9 represents an example model using a convolutional neural network to process an input image for ultrasound-based elastography through shear-wave measurement.

FIG. 10 represents an example system for an ultrasound-based elastography through shear-wave measurement.

FIG. 11 illustrates a flow diagram for a method for an ultrasound-based elastography through shear-wave measurement in accordance with some implementations.

FIG. 12 illustrates a flow diagram for a method for an ultrasound-based elastography through shear-wave measurement in accordance with some implementations.

### DETAILED DESCRIPTION

In order to measure tissue elasticity using an ultrasound system for an ultrasound-based elastography through shear-wave measurement in an anatomy, shear-waves are induced in the anatomy by ultrasound pulses (push pulses). Determination of the shear-wave velocity (vₛ) can, in some examples, be used to determine the tissue elasticity. For example, the Young's modulus can be used as:

$$Y = 3 \cdot ρ {v}_{s}^{2} \qquad Eq . 1$$

In Eq. 1, the Young's modulus (Y) can be used in some examples as an elasticity measure and ρ is a known tissue density. By determining vₛ, the tissue elasticity can be found. Other methods to find the tissue elasticity based on vₛ can be equally employed, giving the more general equation:

$$ϵ = f ( {v}_{s} ) \qquad Eq . 2$$

Eq. 2 shows the tissue density (ϵ) as a function of vₛ(f(vₛ)). Any suitable f(vₛ) can be used, such as Y in Eq. 1. The ultrasound system induces one or more shear waves, such as by ultrasound pulse energy. In aspects, the ultrasound system can acquire electrocardiogram-gated kilohertz visualization (EKV) style data, for example using motion mode (M-mode) ultrasound. In some examples, the M-mode ultrasound is gated by one or more parameters (e.g., patient respiration, echocardiogram, etc.) over a region of interest (ROI).

In some examples, the M-mode ultrasound is synchronized with the push pulses. The push pulses, in some examples, are long-duration, high-power ultrasound pulses. The push pulses can be transmitted along the side of the ROI during each M-mode acquisition (e.g., the pushing aperture position fixed while the M-mode imaging aperture moves across the ROI), or the pushing aperture can move with the imaging aperture. The focused beam used to acquire the M-mode acquisitions increases the performance of the ultrasound system over using other techniques, such as ultrafast plane-wave imaging (e.g., increased signal-to-noise ratio (SNR)). Translation of the pushing aperture in the same way as the imaging aperture moves across the ROI, triggered at each location by the R-wave of the ECG signal, can be used, in some examples, to ensure that, for quickly moving structures (such as the heart), each time-point of the M-mode acquisition (at each lateral location) corresponds to the same phase of the muscle contraction and relaxation cycle.

In some examples, the push pulses can be focused at locations relative to the ROI to induce the shear-wave. The shear wave generation, in aspects, involves multiple push pulses to increase the magnitude of particle displacement caused by the shear wave. The geometric arrangement of the multiple push pulses relative to one another and the anatomy can, in some examples, be used to steer the induced shear-wave. Resultant M-mode data, in aspects, can be reorganized into brightness-mode (B-mode) data. The B-mode data, according to some examples, is high-frame-rate data. In aspects, the B-mode data represents one or more B-mode images.

The B-mode data can be used, in aspects, to determine vₛ. The B-mode data can further be used, in some examples, to estimate, derive, or otherwise generate one or more other parameters relating to the anatomy. For example, the B-mode data can be used to estimate a backscatter coefficient, an attenuation coefficient (AC), etc. The AC can, in some examples, be used for assessing a condition of the anatomy. For example, if the anatomy is a liver of the patient, the AC can be used to assist with discrimination of fatty liver content, steatosis, fibrosis, etc.

Another parameter of the anatomy derived using the B-Mode data, in some examples, can be a speed of sound (SoS) in the anatomy. For example, the SoS can be determined using a two-layer model. In some examples, one layer of the two-layer model is adipose tissue proximal to the liver that is known to have a lower SoS than the liver. In such examples, the second layer can include the liver. A method such as curvature matching of strong scatterers within each layer, in some examples, can be used to estimate or otherwise derive the SoS in the ROI. In some examples, the beamforming can use the SoS and/or the B-mode data as an input parameter in order to direct the beamforming. For example, high-quality B-mode imaging can be performed using a synthetic-transmit-aperture-virtual-source beamformer with the B-mode data and SoS as inputs, allowing for two-stage beamforming. In this case, the first stage beamforming would be used to generate images to track the shear wave propagation and determine the vₛ map; the second stage would be used to enhance the ability to derive other parameters to further discriminate the pathology of the organ undergoing investigation, and/or to enhance the reliability of the determined vₛ measurement and/or the determined vₛ map.

In some examples, the determined vₛ can be determined at multiple locations within the ROI. For example, a directional filtering and cross-correlation method can be used. In some examples, ϵ can be used to find any of the parameters of the anatomy instead of using vₛ. In some examples, the parameters can be found at multiple locations within the ROI.

In some examples, any one or more of the B-mode data, vₛ, ϵ, and the parameters can be used to create an overlay onto a B-mode image output, such as an ultrasound mapping on a display of the ultrasound system. In some examples, the overlay is in the ROI. According to some examples, the overlay is produced by masking certain areas in the ROI. In some examples, the overlay is the product of a machine-learned (ML) model, which can use any one or more of the B-mode data, vₛ, ϵ, and the parameters as inputs. In aspects, the ML model is trained on training data similar to the B-mode data, vₛ, ϵ, and the parameters.

### Ultrasound-Based Elastography Through Shear-Wave Measurement System

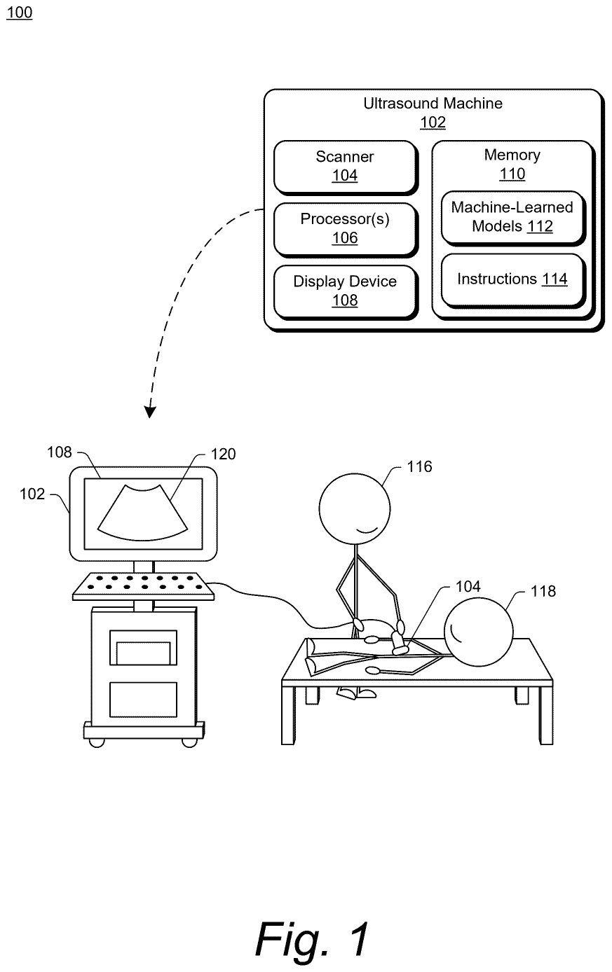

FIG. 1 illustrates an example environment for an ultrasound-based elastography through shear-wave measurement system 100 in accordance with one or more implementations. The shear-wave measurement system 100 is directed to determine an item of interest (e.g., elasticity, speed-of-sound, etc.) within an anatomy. Generally, the shear-wave measurement system 100 includes an ultrasound machine 102, which generates data (including images) based on high-frequency sound waves reflecting off body structures. The ultrasound machine 102 includes various components, some of which include a scanner 104, one or more processors 106, a display device 108, and a memory 110. In an example, the display device 108 can include multiple display devices. In aspects, a first display device can display a first ultrasound image, and a second display device can display a focused ultrasound image or a segmentation image that is generated based on the first ultrasound image. In some implementations, the ultrasound machine 102 also includes one or more ML models 112 and instructions 114 configured to provide input to and/or manipulate output of one or more of the ML models 112.

A user 116 (e.g., nurse, ultrasound technician, operator, sonographer, etc.) directs the scanner 104 toward a patient 118 to non-invasively scan internal bodily structures (e.g., organs, tissues, an anatomy, etc.) of the patient 118 for testing, diagnostic, or therapeutic reasons. In some implementations, the scanner 104 includes an ultrasound transducer array and electronics coupled to the ultrasound transducer array to transmit ultrasound signals to the anatomy of the patient and receive ultrasound signals reflected from the anatomy of the patient. In some implementations, the scanner 104 is an ultrasound scanner, which can also be referred to as an ultrasound probe.

The display device 108 is coupled to the one or more processors 106, which processes the received ultrasound signals to generate ultrasound data. The display device 108 is configured to generate and display an ultrasound image (e.g., ultrasound image 120) of the anatomy based on the ultrasound data generated by the processor 106 from the reflected ultrasound signals detected by the scanner 104. In aspects, the ultrasound data can include data and/or the ultrasound image 120. In some embodiments, the ultrasound data (e.g., the ultrasound image 120, or data representing the ultrasound image 120) is used as input to at least one ML model 112 implemented to identify parts of the anatomy or other anatomy-related aspects scanned by the scanner 104. For example, one ML model 112 can identify one or more organs (including by type) in the ultrasound data and a corresponding location (e.g., position) of each identified organ in the ultrasound data. In another example, the ML model 112 can identify the speed of sound in the anatomy, an elasticity of the anatomy, etc. based on the ultrasound data. In aspects, the ultrasound-based elastography through shear-wave measurement system 100 can determine one or more speed of sound values as described in U.S. patent application Ser. No. 18/542,493 filed Dec. 15, 2023, entitled Ultrasound Parameter Selection Using RF Data to White et al., the disclosure of which is incorporated herein by reference in its entirety.

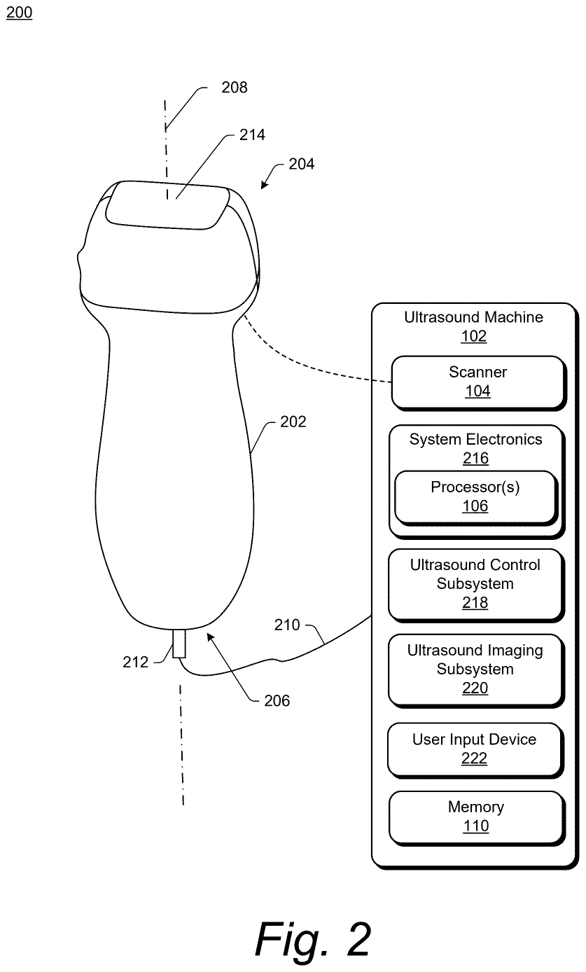

FIG. 2 illustrates an example implementation 200 of the ultrasound machine 102 from FIG. 1. The scanner 104 (e.g., an ultrasound scanner) includes an enclosure 202 extending between a distal end portion 204 and a proximal end portion 206. The enclosure 202 includes a central axis 208 (e.g., longitudinal axis) that intersects the distal end portion 204 and the proximal end portion 206. The central axis 208 corresponds to an axial direction of the scanner 104. In an example, the scanner 104 is electrically coupled to an ultrasound imaging system (e.g., the ultrasound machine 102) via a coupling 210. In implementations, the coupling 210 includes a cable that is attached to the proximal end portion 206 of the scanner 104 by a strain-relief element 212. In some implementations, the coupling 210 includes a wireless electronic coupling so that the scanner 104 is wirelessly coupled to the ultrasound imaging system and communicates with the ultrasound imaging system via one or more wireless transmitters, receivers, or transceivers over a wireless connection or network (Bluetooth™, Wi-Fi™, etc.).

A transducer assembly 214 (e.g., the scanner 104 of FIG. 1) having one or more transducer arrays is electrically coupled to system electronics 216 in the ultrasound machine 102. In operation, the transducer assembly 214 transmits ultrasound energy from the one or more transducer arrays toward a subject and receives ultrasound echoes from the subject. The transmitted ultrasound energy, in some examples, is in the form of one or more ultrasound pulses. The ultrasound echoes are converted into electrical signals by the one or more transducer arrays and electrically transmitted to the system electronics 216 in the ultrasound machine 102 for processing and generation of one or more ultrasound images. In some examples, the transmitted ultrasound energy is in the form of ultrasound pulses. The ultrasound pulses can have parameters, such as waveform, phase, amplitude, target depth, and steering angle.

In some implementations, the system electronics 216 include the one or more processors 106, integrated circuits, application-specific integrated circuits (ASICs), field programmable gate arrays (FPGAs), graphics processing units (GPUs), and power sources to support functioning of the ultrasound machine 102. In some implementations, the ultrasound machine 102 also includes an ultrasound control subsystem 218 having one or more additional processors. At least one processor, FPGA, ASIC, GPU, etc. causes electrical signals to be transmitted to the two or more transducer arrays of the transducer assembly 214 to both emit sound waves and receive electrical pulses from the scanner 104 that were created from the returning echoes. One or more processors, FPGAs, ASICs, GPUs, etc. process the raw data associated with the received electrical pulses and form an image that is sent to an ultrasound imaging subsystem 220, which causes the image to be displayed (e.g., to the display device 108 of the ultrasound machine 102, not pictured in FIG. 2 for clarity). In aspects, the display device 108 displays ultrasound images from the ultrasound data processed by the processor(s) of the ultrasound control subsystem 218.

In some implementations, the ultrasound machine 102 also includes one or more user input devices 222 (a keyboard, a cursor control device, a microphone, a camera, etc.) that input data and enable taking measurements, such as from the display device 108 of the ultrasound machine 102. The ultrasound machine 102 can also include a disk storage device (computer-readable storage media such as read-only memory (ROM), a Flash memory, a dynamic random-access memory (DRAM), a NOR memory, a static random-access memory (SRAM), a NAND memory, etc.) for storing the acquired ultrasound data. In aspects, the disk storage device includes the memory 110 of FIG. 1, which is local to the ultrasound machine 102. Alternatively, the memory 110 used for storing the acquisition data can be remote, such as on a remote server (e.g., 1l medical archiver) communicatively connected to the ultrasound machine 102. In addition, the ultrasound machine 102 can include a printer (not pictured for clarity) that prints the image from the displayed data. To avoid obscuring the techniques described herein, some elements, such as user input devices, a disk storage device, and a printer, are not shown in FIG. 2.

### Shear Wave Generation and Measurement

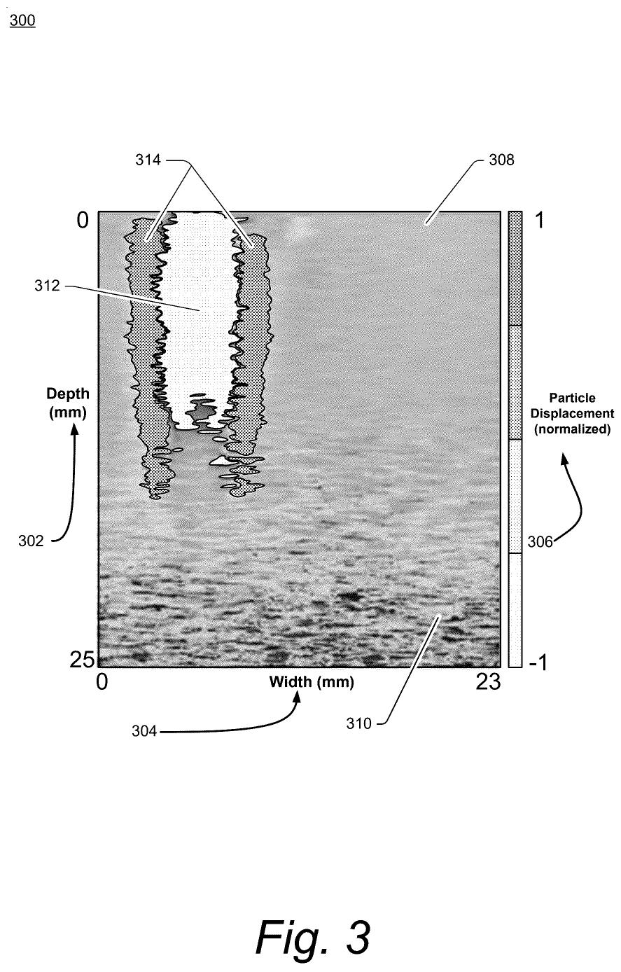

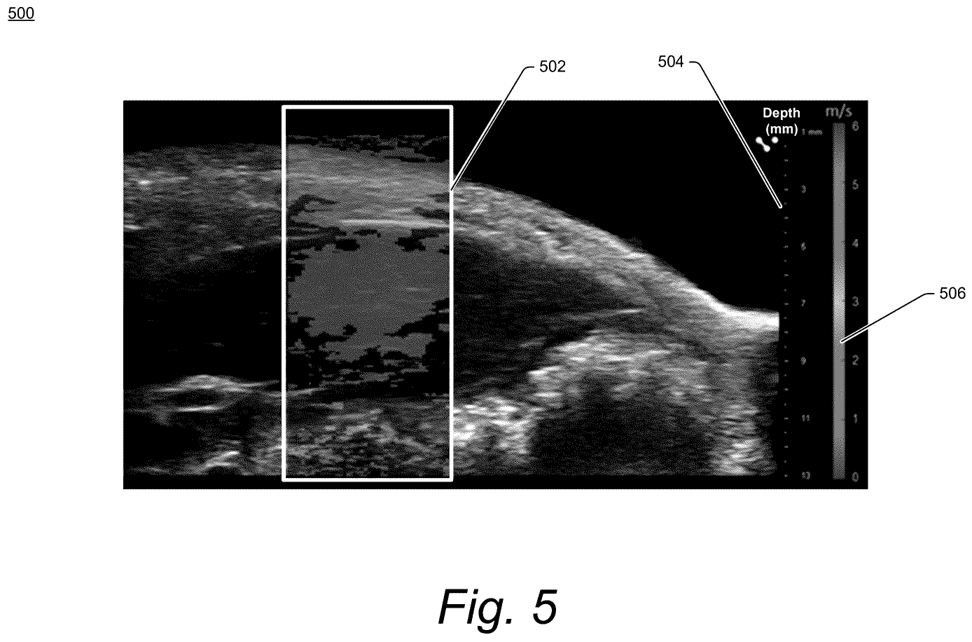

FIG. 3 illustrates a plot 300 of a shear-wave measurement made by an ultrasound system or device for ultrasound-based elastography through shear-wave measurement, such as the ultrasound machine 102 of FIG. 1. The plot 300 can be displayed on a display device, such as the display device 108. In some examples, the plot 300 is not displayed but is plot data of an intermediate processing step, which includes information from which a shear-wave velocity map can be determined and/or generated, such as the example shown in FIG. 5. The plot 300 includes a vertical axis 302 of depth (here shown in millimeters (mm), though this should not be seen as limiting). The plot 300 further includes a horizontal axis 304 of width (here, as in the vertical axis 302, shown in mm, though again this should not be seen as limiting). In some examples and as shown in the plot 300, a color bar 306 (or similar) is used to plot differences in aspects of phenomenon found in the plot 300, here shown as particle displacement. It should be noted that other aspects can be equally employed and/or represented by the color bar 306 or similar, such as, but not limited to, tissue density, temperature, elasticity, etc.

The plot 300 is a plot of an anatomy, such as the anatomy of a patient undergoing an ultrasound examination (e.g., the patient 118 of FIG. 1). The ultrasound machine 102 can, for example, cause ultrasound energy to be incident on the anatomy. Reflections of the ultrasound energy can, in aspects, be received by the ultrasound machine 102, for example by the scanner 104 of FIG. 1. In aspects, the received ultrasound reflections can be used to produce an image, such as the plot 300. Some aspects of the anatomy, in embodiments, can appear on the plot 300 as a background 308 or a noise 310.

According to some embodiments, a shear wave can be induced in the anatomy by the ultrasound energy. For example, an area of indicated low particle displacement 312 can be an area where the ultrasound energy was incident to create the shear wave. The propagation of the shear wave in the anatomy can be monitored using the ultrasound energy by tracking an area of indicated high particle displacement 314. The magnitude of the particle displacement is indicated by the color bar 306.

The shear wave of FIG. 3 can be, in some examples, measured and/or displayed after one or more push pulses are incident upon the anatomy within a region of interest (ROI). In the example plot 300, the one or more push pulses can be incident in an area of the anatomy where the area of indicated low particle displacement 312 is shown. The plot 300 can be made, for example, a short time after the one or more push pulses were incident on the anatomy, such as 20 milliseconds (ms), 30 ms, or another time increment. In some examples, the plot 300 is a dynamic plot, which shows an evolution of the shear wave propagation over a time interval. In such an example, FIG. 3 is a single image sample from a point in time of the time interval.

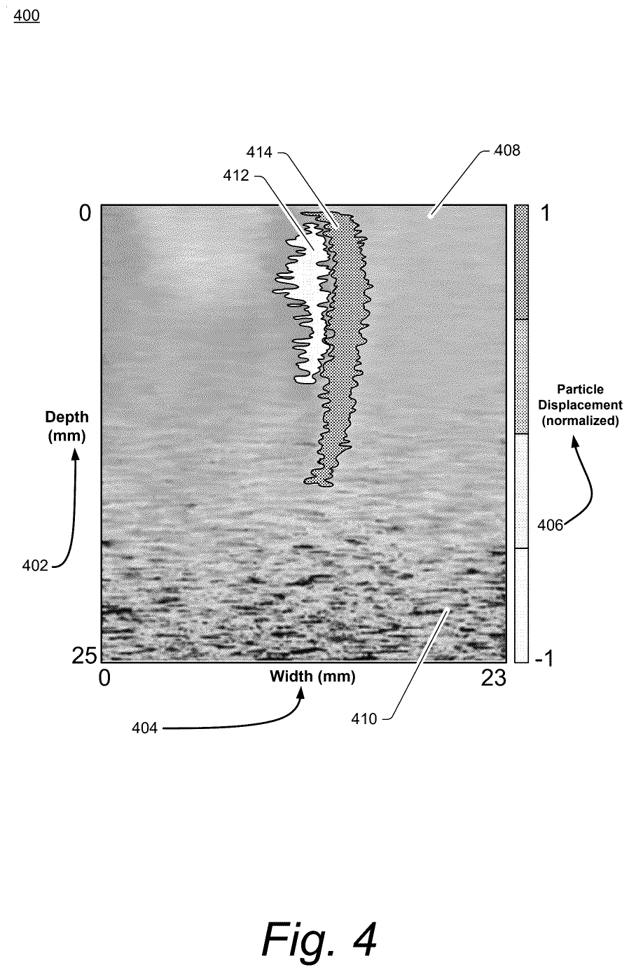

FIG. 4 illustrates a plot 400 showing a measurement of the shear wave of FIG. 3 after a greater time has elapsed than in FIG. 3. For example, the shear wave of FIG. 3 can be measured and/or displayed at a time of 20 ms after one or more push pulses are incident upon an anatomy and the shear wave of FIG. 4 can be measured and/or displayed at a time of 80 ms after the one or more push pulses are incident upon the anatomy. As in FIG. 3, the plot 400 includes a vertical axis 402 of depth (here shown in mm, though this should not be seen as limiting). The plot 400 further includes a horizontal axis 404 of width (here, as in the vertical axis 402, shown in mm, though again this should not be seen as limiting). In some examples and as shown in the plot 400, a color bar 406 (or similar) is used to plot differences in aspects of phenomenon found in the plot 400, here shown as particle displacement. It should be noted that other aspects can be equally employed, such as, but not limited to, tissue density, temperature, elasticity, etc. Some aspects of the anatomy, in aspects, can appear on the plot 400 as a background 408 or a noise 410. In some examples, the plot 400 is not displayed but is plot data, which includes information from which a vₛ map can be determined and/or generated, such as the example shown in FIG. 5.

The plot 400 shows an area of low particle displacement 412 and an area of high vₛ particle displacement 414 (“low” and “high” here are terms relating the various vₛ particle displacements to one another and not necessarily an absolute scale). The area of low vₛ particle displacement 412 and the area of high vₛ particle displacement 414 differ in position and shape from the areas of low vₛ particle displacement 312 and high vₛ particle displacement 314 of FIG. 3. The differences in magnitude, position, etc. between the vₛ values 312, 314, 412, and 414 can, in some examples, be used to determine vₛ, elasticity, etc.

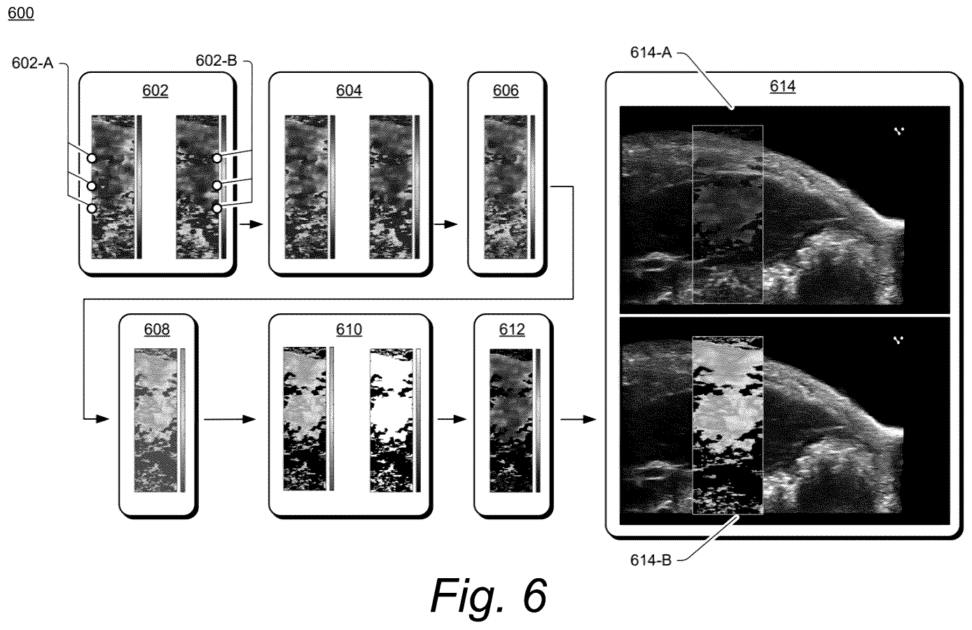

FIG. 5 illustrates an example vₛ map 500 with a region of interest (ROI) 502. Continuing with the liver as an example of the patient anatomy, a user-defined (or ML-determined) ROI 502 within the liver is indicated by a white box. In aspects, an ultrasound system, such as the shear-wave measurement system 100, first applies one or more push pulses (e.g., a series of push pulses) on or outside the left side of the box (e.g., the ROI 502), or inside the box, and the velocity map 500 is calculated. The system then, according to some examples, applies one or more second push pulses on or outside the right side of the box, or inside the box, and another map within the same ROI 502 is calculated by measuring the shear-wave speed propagating in the reverse direction. In aspects, the system then averages and/or combines the maps in any suitable way (described in more detail below with regards to FIG. 6). In embodiments, regions of disagreement are excluded.

The velocity map 500 is shown with a vertical axis 504 indicating depth in mm a color bar 506 indicating shear wave velocity. The ROI 502 indicates a portion of the anatomy. These features of the velocity map 500 are meant to be representative/illustrative and not limiting. For example, the axis 504 can use different measurement units, the color bar 506 can use a different parameter, etc. The ROI 502 can also be larger, smaller, or of a different shape than that illustrated in FIG. 5.

In some examples, the system (e.g., the shear-wave measurement system 100 of FIG. 1) can employ push locations within the ROI 502 in addition to those sent down each side of the ROI 502. The push locations can depend on the size of the ROI 502 and/or a calculated confidence of one or more derived parameter maps (an elasticity-measurement map, a speed-of-sound map, a vₛ map, etc.). For example, consider a shear wave, which propagates well across only half the width of the ROI 502. In such an example, the single shear-wave can be insufficient to provide usable results over the entire width of the ROI 502 and, thus, at least one additional lateral push location located in the ROI 502 can be used to provide additional information. In aspects, a series of additional push pulses can be sent at that same location responsive to a determination that the initial shear wave did not propagate across a large enough portion of the ROI 502. The one or more additional push pulses can be focused at different depths, where the different depths depend on one or more predetermined or derived parameters, such as a vertical size of the ROI 502. A comparison of maps generated from push pulses at different lateral push locations can be used to assess if the number of the lateral push locations is sufficient for ultrasound-based elastography through shear-wave measurement.

In some examples, a push pulse within the ROI 502 can be used when changes in the anatomy occur on a timescale similar to a time it takes for a shear-wave to propagate across a relevant distance. For example, the anatomy can undergo contraction, such as in a heart. Consider, for example, the shear-wave incident upon the left side of the ROI 502 at the same time as the heart is experiencing the contraction and, as the shear wave propagates to the right side of the ROI 502, the contraction ceases. In such an example, parameters related to the shear-wave propagation can change during different portions of the contraction (e.g., E of Eq. 2), and a plurality of push pulses within the ROI 502 can provide imaging and diagnosis advantages.

In aspects, the system determines an averaged map and can overlay it on a B-mode image presented to the user (e.g., via the display device 108 of FIG. 1). In embodiments, the averaged map can also be segmented to exclude vessels within the ROI and tissue outside the target organ based on co-registration with the high-quality B-mode image resulting from the 2-stage synthetic aperture beamforming process.

FIG. 6 depicts a workflow 600 for determining example maps for overlay and user presentation, e.g., via a user interface displayed on the display device 108. In aspects, the system (e.g., the shear-wave measurement system 100 of FIG. 1) first calculates vₛ maps 602 (e.g., the velocity map 500 of FIG. 5) for shear-waves based on push pulses 602-A and 602-B, the shear waves propagating in an anatomy from two or more locations and/or depths. For example, the push pulses 602-A propagate from the left side toward the right side, such that a first vₛ map (e.g., left image of vₛ maps 602) can be generated, and push pulses 602-B propagate from the right side toward the left side, such that a second vₛ map (e.g., right image of vₛ maps 602) can be generated. The system next, in some examples, compares 604 the generated vₛ maps 602 to vₛ threshold values, which can be dependent on aspects of the anatomy being imaged, such as the tissue type. In aspects, the vₛ maps 602 can then be averaged 606 (e.g., combined, summed, weighted, etc.), and a reliability map 608 can be calculated based on differences between the vₛ maps 602.

According to some examples, the reliability map 608 can be used to create one or more threshold maps 610 based on a reliability threshold. In some examples, a reliability threshold may be adjusted by the user. The one or more threshold maps 610, in aspects, are binary masks. The binary masking, in some examples, can be applied to the display of the vₛ maps 602 in areas within an ROI (e.g., the ROI 502 of FIG. 5), such as by displaying areas that meet or exceed the reliability threshold. In some examples, such thresholding and comparison is performed without creating a binary mask or reliability map 608 image but rather consists of a mathematical or data object holding values indicating the threshold-based reliability values. In aspects, the threshold-based reliability values can be determined from a patient history. For example, a tissue density of an anatomy of the patient can be determined from the patient history and a measurement at least in part be based on the tissue density of the anatomy of the patient.

In aspects, the binary mask (or, equivalently, the mathematical or data objects) is applied 612 to the vₛ map 602 and can be used to generate 614 an output for display. For example, an overlay of a Young's Modulus 614-A and/or an overlay of the reliability map 614-B can be displayed to a user on a B-mode image (e.g., the ultrasound image 120). In another embodiment, a display of the reliability map 608 can be shown to the user indicating regions of higher and lower relative reliability of parameters derived or observed from the vₛ mapping and/or B-mode data (e.g., speed-of-sound, elasticity, etc.). In yet another embodiment, the reliability map 608 can be used to trigger a rescan request for areas where the reliability values fall below a threshold value. Such a rescan request can be an output of the rescan request to a user, an automatic rescan performed by the system, generated by machine-learned (ML) model, etc.

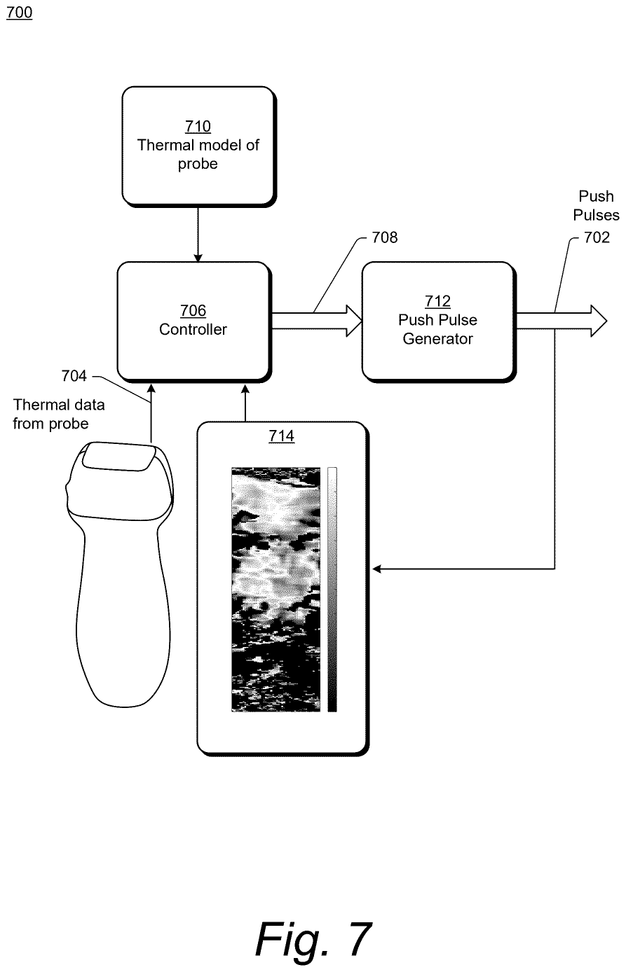

FIG. 7 depicts an example controller 700 to adjust one or more properties of push pulses 702 (e.g., the push pulses 602-A and 602-B of FIG. 6). In aspects of ultrasound-based elastography through shear-wave measurement, thermal data 704 from a probe (e.g., the ultrasound scanner 104 of FIG. 1) is processed by the system (e.g., the shear-wave measurement system 100 of FIG. 1). In some examples, the system implements one or more controllers 706 to adjust one or more properties of the push pulses, as is illustrated in FIG. 7. For instance, the controller 706 can include a feedback controller that includes one or more of a neural network, a proportional-integral-derivative (PID) controller, a state-space controller, etc., and the controller can generate a control signal 708 for adjusting a property of the push pulses 702. For example, the control signal 708 can indicate to the system to adjust (e.g., reduce, increase, etc.) an amplitude, ultrasound frequency, pulse repetition frequency, etc. for the push pulses 702.

In an example, the control signal 708 can indicate to repeat one or more previous push pulses 702, so that the system can re-image a location or generate another map (e.g., the velocity map 500 of FIG. 5) for the location. For instance, the system can determine a low-reliability location based on the reliability map and cause a push pulse generator 712 to generate push pulses 702 to re-image the location, or generate a new vₛ map for the location. The system can then, in aspects, combine the new map for the location with a previous map for other locations to generate a map for an ROI (e.g., the ROI 502 of FIG. 5) that includes the location and the other locations.

Additionally or alternatively, the system can generate the control signal 708 to adjust the push pulses 702 to reduce or maintain a temperature of the probe. The controller 706 can retrieve a thermal model 710 of the probe (e.g., from a database of thermal models for various probes), and, based on the thermal model 710 of the probe and thermal data 704 for the probe in use, generate the control signal 708. The thermal model 710 of the probe can include an equation, table, etc., derived from experimental measurements, such as by pointing a heat gun at the surface of the array of the probe.

Any one or more of the thermal data 704 from the probe, the control signal 708, output from the push pulse generator 712, etc. can be used, in some examples, to generate an output 714. The output 714 can be, for example, a velocity map (e.g., the velocity map 500 of FIG. 5), a stiffness map, a mask overlay, a B-mode image, etc. The output can be displayed, in aspects, by a display of the system (e.g., the display device 108 of FIG. 1). Depending on the type of output, the output can be used, according to some examples, as an input for the controller 706, such as to generate a new control signal 708.

### Machine-Learning

Many of the features described herein can be implemented using a machine-learned model. For the purposes of this disclosure, an ML model is any model that accepts an input (ŝ), analyzes, and/or processes ŝ based on an algorithm derived via machine-learning training, and provides an output (ŷ). The ML model can be conceptualized as a mathematical function of the following form:

$$f ( \hat{s} , θ ) = \hat{y} \qquad Eq . 3$$

In Eq. 3, an operator (f(ŝ,θ)) represents the processing of the ML model based on ŝ and providing ŷ. The term ŝ represents a model input, such as ultrasound data. The model analyzes/processes the s using parameters (θ) to generate ŷ (e.g., a speed-of-sound in an anatomy, an elasticity of the anatomy, etc.). Both ŝ and ŷ can be scalar values, matrices, vectors, or mathematical representations of phenomena such as categories, classifications, image characteristics, the images themselves, text, labels, or the like. The parameters θ can be any suitable mathematical operations, including but not limited to applications of weights and biases, filter coefficients, summations or other aggregations of data inputs, distribution parameters such as mean and variance in a Gaussian distribution, linear-algebra-based operators, or other parameters, including combinations of different parameters, suitable to map data to the desired output.

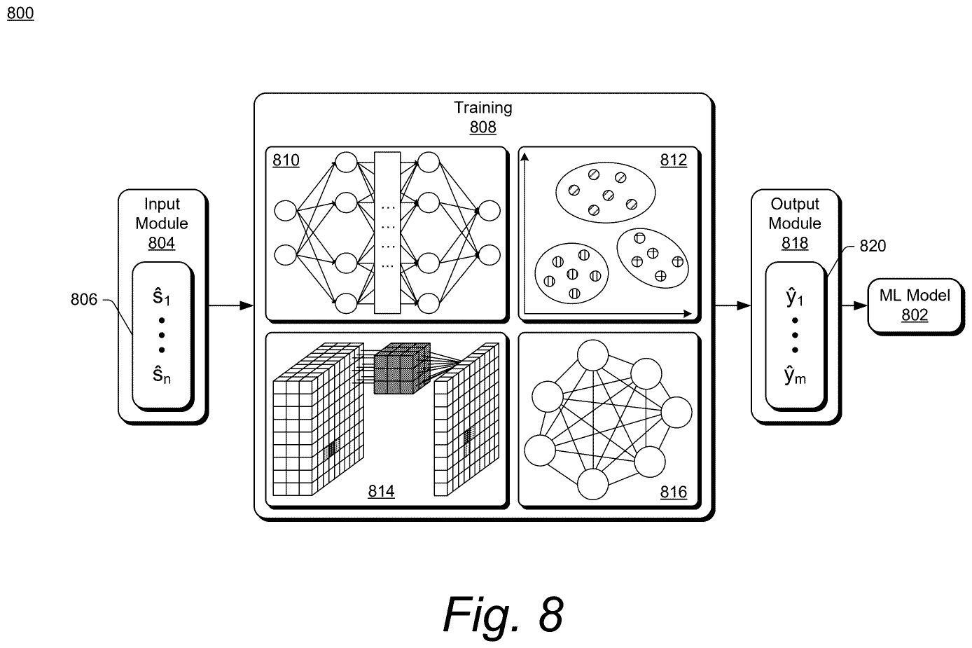

FIG. 8 represents an example machine-learning architecture 800 used to train an ML model M 802. An input module 804 accepts an input (s) 806, which can be an array with members ŝ₁ through ŝₙ. The input s 806 is fed into a training module 808, which processes s 806 based on the machine-learning architecture 800. For example, if the machine-learning architecture 800 uses a multilayer perceptron (MLP) model 810, the training module 808 applies weights and biases to s 806 through one or more layers of perceptrons, each perceptron performing a fit using its own weights and biases according to its given functional form. MLP weights and biases can be adjusted so that they are optimized against a least mean square, logcosh, or other optimization function (e.g., loss function) known in the art. Although an MLP model 810 is described here as an example, any suitable machine-learning technique can be employed, some examples of which include but are not limited to k-means clustering 812, convolutional neural networks (CNN) 814, a Boltzmann machine 816, Gaussian mixture models (GMM), and long short-term memory (LSTM). The training module 808 provides an input to an output module 818. The output module 818 analyzes the input from the training module 808 and provides a prediction output (ŷ) 820, which can be an array with members ŷ₁ through ŷₘ. The prediction output f 820 can represent a known correlation with the input ŝ 806 (e.g., the velocity map 500 of FIG. 5, the ROI 502 of FIG. 5, the threshold map 610 of FIG. 6, etc.).

In some examples, ŝ 806 can be training input labeled with known output correlation values, and these known values can be used to optimize ŷ 820 in training against the optimization/loss function. In other examples, the machine-learning architecture 800 can categorize ŷ 820 values without being given known correlation values to ŝ 806. In some examples, the machine-learning architecture 800 can be a combination of machine-learning architectures. By way of example, a first network can use ŝ 806 and provide ŷ 820 as a second input (ŝ_{ML}) to a second ML architecture (not pictured), with the second machine-learning architecture providing a final prediction output (ŷ_{f}). In another example, one or more machine-learning architectures can be implemented at various points throughout the training module 808.

In some ML models, all layers of the model are fully connected. For example, all perceptrons in an MLP model act on every member of s 806. For example, an MLP model with a 100×100 pixel image as the input, each perceptron provides weights/biases for 10,000 inputs. With a large, densely layered model, this can result in slower processing and/or issues with vanishing and/or exploding gradients. A CNN, which can be a non-fully connected model (including a fully disconnected model), can process the same image using 5×5 tiled regions, requiring only 25 perceptrons with shared weights, giving much greater efficiency than the fully connected MLP model.

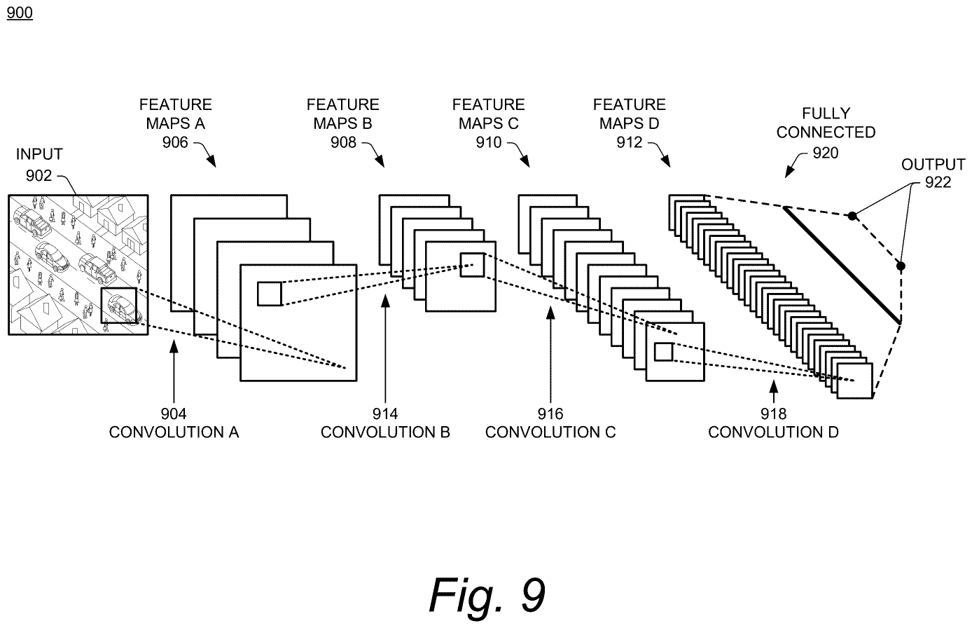

FIG. 9 represents an example model 900 using a CNN to process an input 902, which, in the example shown in FIG. 9, includes representations of objects that can be identified via object recognition, such as people or cars. Although this example includes people and cars as general objects in the input 902, the input 902 can include an ultrasound image, as described above, having representations of anatomy, such as bodily structures. The input 902 can be, in some examples, an image, data representative of an image, or other data, such as ultrasound data. Convolution A 904 can be performed to create a first set of feature maps (e.g., feature maps A 906). A feature map can be a mapping of aspects of the input 902 given by a filter element of the CNN. This process can be repeated using feature maps A 906 to generate further feature maps B 908, feature maps C 910, and feature maps D 912 using convolution B 914, convolution C 916, and convolution D 918, respectively. In this example, feature maps D 912 become the input for fully connected network layers 920. In this way, the ML model 900 can be trained to recognize certain elements of the image, such as people or cars, and provide an output 922 that, for example, identifies the recognized elements. For example, the model 900 can be used to identify parts of the anatomy (e.g., organs, vessels, tumors, fluids, etc.).

Although the example of FIG. 9 shows the CNN as a part of a fully connected network, other architectures are possible, and this example should not be seen as limiting. There can be more or fewer layers in the CNN. A CNN component for a model can be placed in a different order, or the model can contain additional components or models. There can be no fully connected components, such as a fully disconnected network. Additional aspects of the CNN, such as pooling, downsampling, upsampling, or other aspects known to people skilled in the art can also be employed.

### Example Device

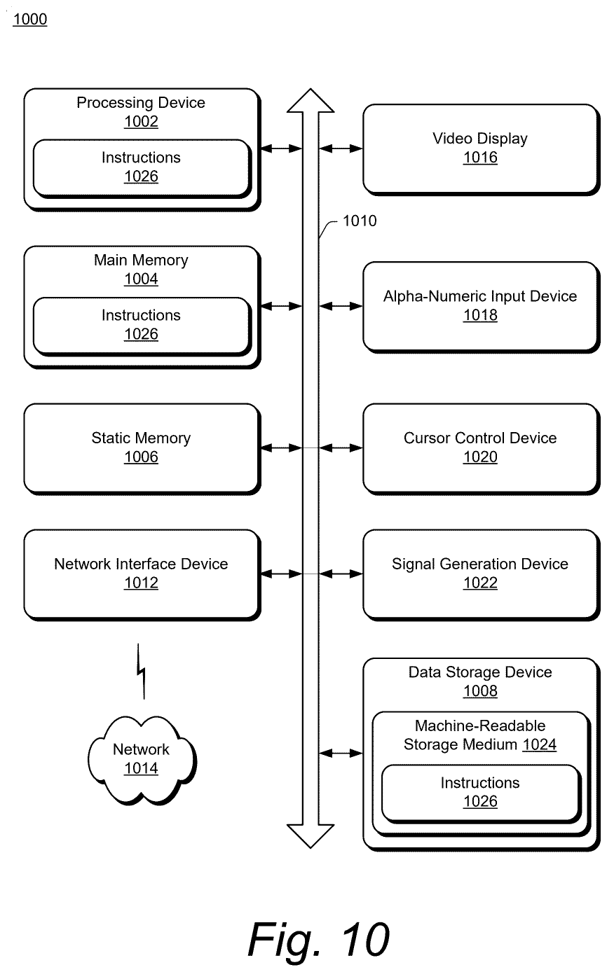

FIG. 10 illustrates a block diagram of an example computing device 1000 that can perform one or more of the operations described herein, in accordance with some implementations. The computing device 1000 can be connected to other computing devices in a LAN, an intranet, an extranet, and/or the Internet. The computing device can operate in the capacity of a server machine in a client-server network environment or in the capacity of a client in a peer-to-peer network environment. The computing device can be provided by a personal computer (PC), a server computer, a desktop computer, a laptop computer, a tablet computer, a smartphone, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein. In some implementations, the computing device 1000 is one or more of an ultrasound machine, an access point, and a packet-forwarding component.

The example computing device 1000 can include a processing device 1002 (e.g., a general-purpose processor, a PLD, etc.), a main memory 1004 (e.g., synchronous dynamic random-access memory (DRAM), read-only memory (ROM)), and a static memory 1006 (e.g., flash memory and a data storage device 1008), which can communicate with each other via a bus 1010. The processing device 1002 can be provided by one or more general-purpose processing devices such as a microprocessor, a central processing unit, or the like. In an illustrative example, the processing device 1002 comprises a complex instruction set computing (CISC) microprocessor, a reduced instruction set computing (RISC) microprocessor, a very long instruction word (VLIW) microprocessor, or a processor implementing other instruction sets or processors implementing a combination of instruction sets. The processing device 1002 can also comprise one or more special-purpose processing devices such as an ASIC, an FPGA, a digital signal processor (DSP), a network processor, or the like. The processing device 1002 can be configured to execute the operations described herein, in accordance with one or more aspects of the present disclosure, for performing the operations and steps discussed herein.

The computing device 1000 can further include a network interface device 1012, which can communicate with a network 1014. The computing device 1000 also can include a video display unit 1016 (e.g., a liquid crystal display (LCD), organic light-emitting diode (OLED), or a cathode ray tube (CRT)), an alphanumeric input device 1018 (e.g., a keyboard), a cursor control device 1020 (e.g., a mouse), and an acoustic signal generation device 1022 (e.g., a speaker and/or a microphone). In one embodiment, the video display unit 1016, the alphanumeric input device 1018, and the cursor control device 1020 can be combined into a single component or device (e.g., an LCD touch screen).

The data storage device 1008 can include a computer-readable storage medium 1024 on which can be stored one or more sets of instructions 1026 (e.g., instructions for carrying out the operations described herein, in accordance with one or more aspects of the present disclosure). The instructions 1026 can also reside, completely or at least partially, within the main memory 1004 and/or within the processing device 1002 during execution thereof by the computing device 1000, where the main memory 1004 and the processing device 1002 also constitute computer-readable media. The instructions can further be transmitted or received over the network 1014 via the network interface device 1012.

Various techniques are described in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. In some aspects, the modules described herein (e.g., the input module 804, the training module 808, and the output module 818) are embodied in the data storage device 1008 of the computing device 1000 as executable instructions or code. Although represented as software implementations, the described modules can be implemented as any form of a control application, software application, signal-processing and control module, hardware, or firmware installed on the computing device 1000.

While the computer-readable storage medium 1024 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding, or carrying a set of instructions for execution by the machine and that causes the machine to perform the methods described herein. The term “computer-readable storage medium” shall accordingly be taken to include, but not be limited to, solid-state memories, optical media, and magnetic media.

### Example Method

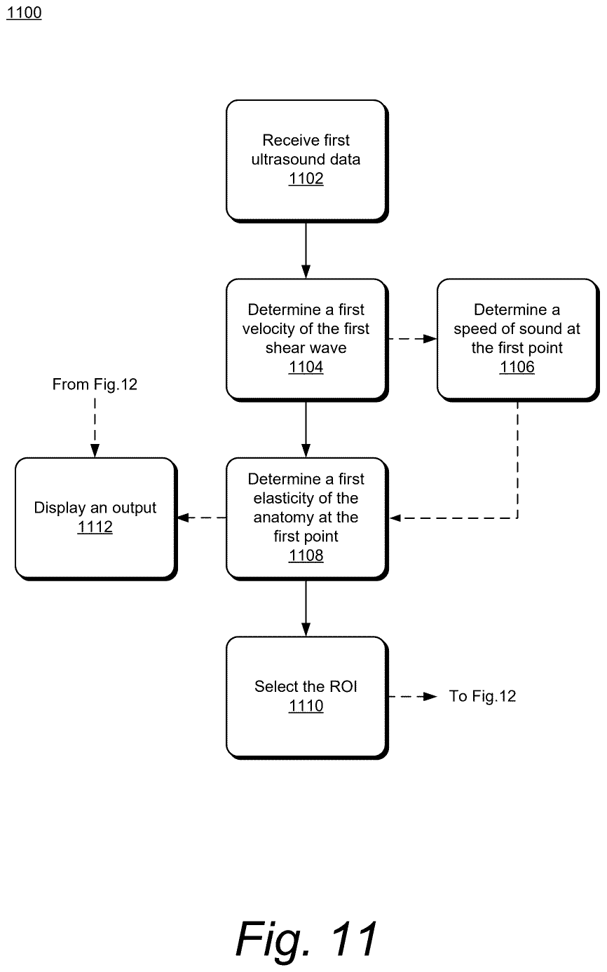

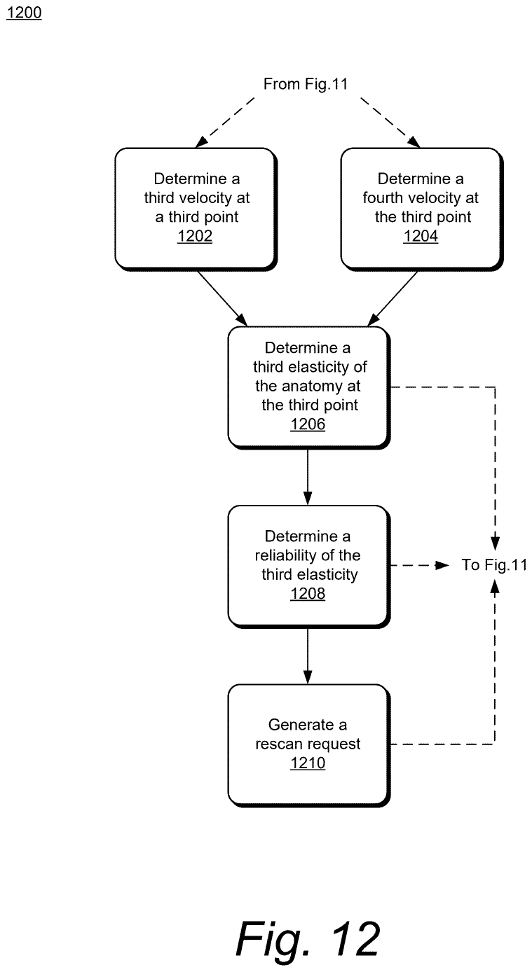

FIGS. 11 and 12 depict flow diagrams for methods 1100 and 1200, respectively, for ultrasound-based elastography through shear-wave measurement in accordance with some implementations. The methods 1100 and 1200 are shown as a set of blocks that specify operations performed but are not necessarily limited to the order or combinations shown for performing the operations by the respective blocks. Further, any of one or more of the operations can be repeated, combined, reorganized, or linked to provide a wide array of additional and/or alternate methods. For example, the method 1200 can be combined with the method 1100 to form a single method, or the methods 1100 and 1200 can be performed separately and individually. In portions of the following discussion, reference can be made to the shear-wave measurement system 100 of FIG. 1 or to entities or processes as detailed in FIGS. 2-10, reference to which is made for example only. The techniques are not limited to performance by one entity or multiple entities operating on one device. The methods 1100 and 1200 can be performed by an ultrasound machine, such as the ultrasound machine 102 of FIG. 1, as described herein.

At 1102, first ultrasound data is received. In aspects, the first ultrasound data can be based on reflections of ultrasound signals transmitted by an ultrasound scanner (e.g., the scanner 104 of FIG. 1) at an anatomy, such as an anatomy of a patient (e.g., the patient 118 of FIG. 1). The first ultrasound data, in aspects, is based on a first push pulse transmitted by the ultrasound scanner at a first point within an ROI (e.g., the ROI 502 of FIG. 5). The first push pulse, in aspects, induces a first shear wave within the anatomy. In some examples, the first push pulse may be a set of push pulses and include multiple push pulses (e.g., the first push pulses 602-A of FIG. 6), such as a superposition of first push pulses. In some examples, the first shear-wave includes a displacement of the anatomy at the first point.

In some examples, the first push pulse is beamformed, steered, or otherwise directed. In some examples, the ultrasound data is based on both the shear wave and other ultrasound signals reflected from the anatomy. For example, the ultrasound data can be based on both the first push pulses and ultrasound signals used to generate an ultrasound image, such as an M-mode or B-mode image. Other combinations of ultrasound data based on the first push pulses and additional ultrasound data are also possible (e.g., A-mode ultrasound data, doppler mode ultrasound data, etc.).

At 1104, a first velocity of the first shear-wave is determined. In aspects, the determination of the first velocity is based on one or more of the first shear wave, the first push pulse or set of push pulses, and the first ultrasound data. At 1106, in some examples, a speed of sound is determined at the first point, for example based on B-mode data vₛ. In aspects, the determined speed of sound can be different than the determined vₛ. In some examples, the determination of the speed of sound is performed by an ML model (e.g., the ML model 802 of FIG. 8, the model 900 of FIG. 9, etc.).

At 1108, a first elasticity of the anatomy at the first point is determined. In aspects, the first elasticity is based on the first velocity of the first shear-wave. In some examples, the first elasticity is further based on the speed of sound at the first point. In some examples, the determination of the first elasticity is performed by the ML model. According to some examples, the ML model takes one or more of the determined first velocity of the first shear-wave and the determination of the speed of sound at the first point as inputs.

At 1110, an ROI is selected (e.g., the ROI 502 of FIG. 5). In some examples, the selection of the ROI is performed by the ML model. In some examples, a user (e.g., the user 116 of FIG. 1) selects the ROI. In other examples, the ROI is a default ROI.

At 1112, an output is displayed. In some examples, the output is based on the first elasticity. In some examples, the output is based on the determined vₛ. In some examples, the output is a map, e.g., an elasticity-measurement map, a vₛ map, etc.

FIG. 12 illustrates the flow diagram for the method 1200 for an ultrasound-based elastography through shear-wave measurement in accordance with some implementations. In aspects, a method outlined by the method 1100 of FIG. 11 is performed, at least inclusive of 1102, 1104, and 1108. In aspects, the method outlined by the method 1100 of FIG. 11 is performed twice at two distinct points in the anatomy, resulting in the determined first velocity of the first shear-wave at the first point and a second velocity of a second shear-wave at a second point in the anatomy (based on the first ultrasound data and second ultrasound data, respectively), the second point being different than the first point. At 1202, a third velocity is determined at a third point in the anatomy, the determination of the third velocity based on the first ultrasound data and the third point being different than both the first and the second points.

At 1204, a fourth velocity is determined at the third point in the anatomy, the determination of the fourth velocity based on the second ultrasound data. At 1206, a third elasticity of the anatomy at the third point is determined based on the third velocity and the fourth velocity. In some examples, the third elasticity is used as an output for display, as in 1112 or FIG. 11. In some examples, the determination of the third elasticity is performed by an ML model (e.g., the ML model 802 of FIG. 8, the model 900 of FIG. 9, etc.).

At 1208, a reliability of the third elasticity is determined. In aspects, the determination of the reliability of the third elasticity is based on the third velocity and the fourth velocity. In some examples, the determined reliability is used as an output for display, as in 1112 or FIG. 11. In some examples, the determination of the reliability of the third elasticity is performed by the ML model.

At 1210, a rescan request is generated. In aspects, the generation of the rescan request is based on the determined reliability of the third elasticity not exceeding or meeting a reliability threshold. In some examples, the generated rescan request includes a command to perform an ultrasound scan at the third point in the anatomy, such as by generating a third ultrasound data in a manner similar to that outlined in 1102 of FIG. 11. In some examples, the generation of the rescan request is used as an output for display, as in 1112 or FIG. 11. In some examples, the generation of the rescan request is performed by the ML model.

### Additional Examples

Example 1: A method for measuring an elasticity value for an anatomy using ultrasound, the method including receiving first ultrasound data based on reflections of ultrasound signals transmitted by an ultrasound scanner at the anatomy. The first ultrasound data is based on a first push pulse transmitted by the ultrasound scanner at a first point within a region of interest (ROI). The ROI is a region of the anatomy. The first push pulse is configured to induce a first shear wave within the anatomy. The method further includes determining, by one or more processors and based on the first push pulse, a first velocity of the first shear wave. The method further includes determining, by the one or more processors and based on the first velocity, a first elasticity of the anatomy at the first point.

Example 2: The method of example 1, further including selecting, by the one or more processors, the ROI.

Example 3: The method of example 2, where the selection of the ROI is performed using a machine-learned model.

Example 4: The method of example 1, further including receiving second ultrasound data based on a second push pulse transmitted by the ultrasound scanner at a second point within the ROI, the second push pulse configured to induce a second shear wave within the anatomy. The method further includes determining, by the one or more processors and based on the second push pulse, a second velocity of the second shear wave. The method further includes determining, by the one or more processors and based on the second velocity, a second elasticity of the anatomy at the second point. The method further includes and generating, by the one or more processors, an output of the first elasticity of the anatomy at the first point and the second elasticity of the anatomy at the second point.

Example 5: The method of example 4, further including displaying, by a display element, the output of the first elasticity of the anatomy at the first point and the second elasticity of the anatomy at the second point.

Example 6: The method of claim 4, further including determining, by the one or more processors and based on the first push pulse, a third velocity at a third point in the anatomy. The third point is different than the first point and the second point. The method further includes determining, by the one or more processors and based on the second push pulse, a fourth velocity at the third point in the anatomy.

Example 7: The method of example 6, further including determining, by the one or more processors and based on the third velocity and the fourth velocity, a third elasticity of the anatomy at the third point.

Example 8: The method of example 7, further including determining, by the one or more processors and based on the third velocity and the fourth velocity, a reliability of the third elasticity of the anatomy at the third point. The method further includes determining, by the one or more processors, whether the reliability of the third elasticity of the anatomy at the third point exceeds a reliability threshold value.

Example 9: The method of example 8, further including displaying, by a display element, the reliability of the third elasticity of the anatomy at the third point.

Example 10: The method of example 8, further including generating, by the one or more processors and based on the determination that the reliability of the third elasticity of the anatomy at the third point is not exceeding the reliability threshold value, a rescan request for the third point.

Example 11: The method of example 8, where the determination of the reliability of the third elasticity of the anatomy at the third point is performed using a machine-learned model.

Example 12: The method of example 10, further including generating, by the one or more processors, an output of the rescan request.

Example 13: The method of example 12, further including displaying, by a display element, the output of the rescan request.

Example 14: The method of example 1, where the ultrasound scanner is further configured to beamform the ultrasound signals transmitted by the ultrasound scanner at the anatomy.

Example 15: The method of example 1, where the first push pulse includes a plurality of constituent push pulses, the constituent push pulses configured such that a superposition of the constituent push pulses form a quasi-planar shear wave, the first push pulse including the planar shear wave.

Example 16: The method of example 1, further including determining, by the one or more processors and based on the first ultrasound data, a speed of sound at the first point in the anatomy.

Example 17: The method of example 16, where the determination of the first elasticity is performed using a machine-learned model. One or more of the first velocity, the first ultrasound data, or the determined speed of sound at the first point in the anatomy are used as inputs for the machine-learned model.

Example 18: The method of example 16, where the determination of the speed of sound at the first point in the anatomy is performed using a machine-learned model.

Example 19: The method of example 1, where the determination of the first elasticity is performed using a machine-learned model.

Example 20: The method of example 1, where the first shear wave includes a displacement of the anatomy at the first point.

Example 21: An ultrasound system including an ultrasound scanner, the ultrasound system configured to execute any one of the methods 1-20.

Example 22: A non-transitory, computer-readable medium that, when accessed by one or more processors, causes the one or more processors to perform any one of the methods 1-20.

Example 23: A computer-program product that, when accessed by one or more processors, causes the one or more processors to perform any one of the methods 1-20.

### CONCLUSION

Embodiments of ultrasound-based elastography through shear-wave measurement as described herein are advantageous, as they can enhance performance of a measurement by an ultrasound system of parameters relevant to an ultrasound examination, such as anatomy elasticity and the speed-of-sound within the anatomy, which can help with diagnosis and thereby improve care provided to a patient. The ultrasound-based elastography through shear-wave measurement can also generate reliability ratings for these parameter values, as well as indicate, based on the reliability values, if there are areas of the anatomy, which require rescanning. Various wave- and beam-forming techniques as described herein can be employed to increase speed and/or reliability of elasticity and shear wave velocity measurements taken with the ultrasound system.

1. An ultrasound system comprising:
an ultrasound scanner configured to:
transmit ultrasound signals at an anatomy; and
generate a first push pulse at a first point within a region of interest (ROI), the ROI comprising a region of the anatomy, the first push pulse configured to induce a first shear wave within the anatomy;
one or more processors in communication with the ultrasound scanner; and
one or more computer-readable storage media having instructions stored thereon that, responsive to execution by the one or more processors, cause the one or more processors to:
determine, based on the first push pulse and reflections of the ultrasound signals transmitted at the anatomy, a first velocity of the first shear wave;
provide the first velocity as an input to a machine-learned model; and
receive, from the machine-learned model and based on the first velocity, a first elasticity of the anatomy at the first point.

2. The ultrasound system of claim 1, wherein the instructions further cause the one or more processors to select the ROI.

3. The ultrasound system of claim 2, wherein the selection of the ROI is performed using the machine-learned model.

4. The ultrasound system of claim 1, wherein:
the input is a first input;
the ultrasound scanner is further configured to generate a second push pulse at a second point within the ROI, the second push pulse configured to induce a second shear wave within the anatomy; and
the instructions further cause the one or more processors to:
determine, based on the second push pulse and the reflections of the ultrasound signals, a second velocity of the second shear wave;
provide the second velocity as a second input to a machine-learned model; and
receive, from the machine-learned model and based on the second velocity, a second elasticity of the anatomy at the second point.

5. The ultrasound system of claim 4, wherein:
the instructions further cause the one or more processors to generate an output of the first elasticity of the anatomy at the first point and the second elasticity of the anatomy at the second point; and
the ultrasound system further comprises a display element, wherein the display element is configured to display the output of the first elasticity of the anatomy at the first point and the second elasticity of the anatomy at the second point.

6. The ultrasound system of claim 4, wherein the instructions further cause the one or more processors to:
determine, based on the first push pulse and the reflections of the ultrasound signals, a third velocity at a third point in the anatomy, the third point being different than the first point and the second point; and
determine, based on the second push pulse and the reflections of the ultrasound signals, a fourth velocity at the third point in the anatomy.

7. The ultrasound system of claim 6, wherein the instructions further cause the one or more processors to determine, based on the third velocity and the fourth velocity, a third elasticity of the anatomy at the third point.

8. The ultrasound system of claim 7, wherein the instructions further cause the one or more processors to:
determine, based on the third velocity and the fourth velocity, a reliability of the third elasticity of the anatomy at the third point; and
determine whether the reliability of the third elasticity of the anatomy at the third point exceeds a reliability threshold value.

9. The ultrasound system of claim 8, further comprising a display element, wherein the display element is configured to display the reliability of the third elasticity of the anatomy at the third point.

10. The ultrasound system of claim 8, wherein the instructions further cause the one or more processors to, based on the determination that the reliability of the third elasticity of the anatomy at the third point is not exceeding the reliability threshold value, generate a rescan request for the third point.

11. The ultrasound system of claim 8, wherein the determination of the reliability of the third elasticity of the anatomy at the third point is performed using the machine-learned model.

12. (canceled)

13. The ultrasound system of claim 1, wherein the first shear wave comprises a displacement of the anatomy at the first point.

14. A method for measuring an elasticity value for an anatomy using ultrasound, the method comprising:
receiving first ultrasound data based on reflections of ultrasound signals transmitted by an ultrasound scanner at the anatomy, the first ultrasound data based on a first push pulse transmitted by the ultrasound scanner at a first point within a region of interest (ROI), the ROI comprising a region of the anatomy and the first push pulse configured to induce a first shear wave within the anatomy;
determining, by one or more processors and based on the first push pulse, a first velocity of the first shear wave;
providing the first velocity as an input to a machine-learned model; and
receiving, from the machine-learned model, by the one or more processors, and based on the first velocity, a first elasticity of the anatomy at the first point.

15. The method of claim 14, further comprising selecting, by the one or more processors, the ROI.

16. The method of claim 15, wherein the selection of the ROI is performed using the machine-learned model.

17. The method of claim 14, wherein the input is a first input, the method further comprising:
receiving second ultrasound data based on a second push pulse transmitted by the ultrasound scanner at a second point within the ROI, the second push pulse configured to induce a second shear wave within the anatomy;
determining, by the one or more processors and based on the second push pulse, a second velocity of the second shear wave;
providing the second velocity as a second input to the machine-learned model;
receiving, from the machine-learned model, by the one or more processors, and based on the second velocity, a second elasticity of the anatomy at the second point; and
generating, by the one or more processors, an output of the first elasticity of the anatomy at the first point and the second elasticity of the anatomy at the second point.

18. The method of claim 17, further comprising displaying, by a display element, the output of the first elasticity of the anatomy at the first point and the second elasticity of the anatomy at the second point.

19. The method of claim 17, further comprising:
determining, by the one or more processors and based on the first push pulse, a third velocity at a third point in the anatomy, the third point different than the first point and the second point; and
determining, by the one or more processors and based on the second push pulse, a fourth velocity at the third point in the anatomy.

20. The method of claim 19, further comprising determining, by the one or more processors and based on the third velocity and the fourth velocity, a third elasticity of the anatomy at the third point.

21. The method of claim 20, further comprising:
determining, by the one or more processors and based on the third velocity and the fourth velocity, a reliability of the third elasticity of the anatomy at the third point; and
determining, by the one or more processors, whether the reliability of the third elasticity of the anatomy at the third point exceeds a reliability threshold value.
